(19) United States
(12) Patent Application Publication
(10) Pub. No.: US 2025/0064621 A1
(43) Pub. Date: Feb. 27, 2025

(54) Neck Relief Arm Sling Adapter

(71) Applicant: James Talley Abel, Pocatello, ID (US)

(72) Inventor: James Talley Abel, Pocatello, ID (US)

(21) Appl. No.: 18/770,373

(22) Filed: Jul. 11, 2024

Related U.S. Application Data
(60) Provisional application No. 63/520,871, filed on Aug. 21, 2023.

(51) Int. Cl. A61F 5/37; A45F 3/00; A45F 3/02

(52) U.S. Cl. CPC A61F 5/3738; A45F 3/02; A45F 2003/003

(57) ABSTRACT

An apparatus and technique provide a sling with a support sleeve, or a support sleeve for a sling device such as an arm sling or an accessory such as a shoulder bag. The support sleeve includes a sleeve portion that is wrapped around a user's arm, to support the weight of the opposite slinged arm or the accessory. One or more slide attachments are permanently or removably attached to the sleeve portion, so as to engage the strap of the sling or the accessory bag.

### PRIORITY CLAIM AND CROSS-REFERENCE TO RELATED APPLICATION

This application claims the benefit under 35 U.S.C. § 119 (e) (1) of U.S. Provisional Application No. 63/520,871, filed Aug. 21, 2023, which is hereby incorporated by reference in its entirety.

### BACKGROUND

Devices such as arm slings and the like may be used to support, immobilize, or restrict movement of an arm, for example. Arm slings may be worn by persons with wrist, arm, or shoulder injuries, for instance, to help prevent further injury and to promote healing. Using an arm sling can also help manage the pain associated with an injury by supporting the weight of the injured or associated arm.

Most arm slings and like devices include a pocket portion and a shoulder/neck strap. The injured arm (or the arm associated with the injured shoulder, wrist, etc.) is cradled in the pocket portion, which generally comprises a cloth pouch or a similar device. The shoulder/neck strap is commonly attached to the pocket portion at two points-near the wrist/hand opening of the pocket portion and near the elbow area of the pocket portion. The shoulder/neck strap is looped around the neck of the user, to an opposite side of the user's neck from the slinged arm, such that the weight of the slinged arm within the pocket portion is initially supported by the shoulder or the neck of the user.

In such an arrangement, the force applied by the weight of the slinged arm is directed downward and inward (toward the pocket portion and toward the user's neck). Accordingly, even when the shoulder/neck strap is initially placed closer to the user's shoulder, the inward pulling force applied by the weight of the slinged arm tends to move the shoulder/neck strap closer to the user's neck as the sling is worn over a short period of time. This shoulder/neck strap movement shifts increasingly more of the weight of the slinged arm to the user's neck, and can cause the user neck pain, neck injury, and related discomfort.

Similarly, when a user is carrying a shoulder bag, purse, messenger bag, duffel bag, or other accessory that has a strap that goes over the user's shoulder and neck in a similar manner (particularly cross-body), the force applied by the weight of the accessory is directed downward and inward toward the user's neck. The inward pulling force applied by the weight of the accessory tends to move the shoulder/neck strap closer to the user's neck and the shoulder/neck strap shifts increasingly more of the weight of the accessory to the user's neck, also causing neck pain, neck injury, and related discomfort. Instead of the weight of the load being carried on the user's shoulder and back, the weight is carried on the user's neck, which can be highly uncomfortable for the user and can also be damaging to muscles and soft tissues of the user's neck, including to the user's nerves and blood vessels.

In some cases, a shoulder/neck strap may be padded to improve the comfort of wearing the arm sling or accessory over time. For example, soft padding can be located on the shoulder/neck strap and positioned at the user's neck. However, while the soft padding may reduce some discomfort associated with the shoulder/neck strap rubbing on the user's neck (causing chafing, abrasions, etc. of the skin on the user's neck), the soft padding does not reduce the amount of force applied to the user's neck by the weight of the slinged arm or accessory. Consequently, neck pain, fatigue, headaches, and neck injury may still occur with prolonged use of the arm sling or accessory.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items.

For this discussion, the devices and systems illustrated in the figures are shown as having a multiplicity of components. Various implementations of devices and/or systems, as described herein, may include fewer components and remain within the scope of the disclosure. Alternately, other implementations of devices and/or systems may include additional components, or various combinations of the described components, and remain within the scope of the disclosure. Shapes and/or dimensions shown in the illustrations of the figures are for example, and other shapes and or dimensions may be used and remain within the scope of the disclosure, unless specified otherwise.

FIG. 1 is a front view of a user wearing an example sling device.

FIG. 2 is a perspective view of a user wearing an example sling device with a support sleeve, according to an embodiment.

FIG. 3 is a side view of a user wearing an example sling device with a support sleeve, according to an embodiment.

FIG. 4 is a front view of an example support sleeve, according to an embodiment.

FIGS. 5A and 5B show two perspective views of an example support sleeve, according to an embodiment.

FIGS. 6A and 6B show two profile views of example support sleeves, according to various embodiments.

FIGS. 7A-7C show three views of an example support sleeve, according to an embodiment.

FIGS. 8A and 8B show two views of an example support sleeve, according to another embodiment.

FIG. 9A shows a side view of an example support sleeve, according to another embodiment.

FIG. 9B shows a perspective view of a user wearing an example sling device with a support sleeve of FIG. 9A, according to an embodiment.

FIG. 10A shows a side view of an example support sleeve, according to another embodiment.

FIG. 10B shows a perspective view of a user wearing an example sling device with a support sleeve of FIG. 10A, according to an embodiment.

FIG. 11 shows a perspective view of a user wearing an example sling device with a support sleeve, according to an embodiment.

### DETAILED DESCRIPTION

### Introduction

Representative implementations of devices and techniques provide a sling with a support sleeve, or a support sleeve for a sling device such as an arm sling or an accessory such as a shoulder bag. The support sleeve includes a sleeve portion that is wrapped around a user's arm, to support the weight of the opposite slinged arm or an accessory bag (or the like). One or more slide attachments are associated with the sleeve portion. In one embodiment, a slide attachment is attached to an upper part of the sleeve portion, so as to be disposed near a shoulder of the user. The slide attachment(s) are slideably coupled to a shoulder strap (for instance) of the sling device or accessory, and may have freedom to move with respect to the shoulder strap.

In various implementations, the support sleeve positions the shoulder strap of the sling device or accessory on the user, and maintains the shoulder strap in a desired location and position. For example, the support sleeve can maintain the shoulder strap at a preferred location on the user's shoulder, even while the sling device or accessory is worn over a prolonged period of time. Positioning and maintaining the shoulder strap in the desired location and position can shift the force applied by the weight of the slinged arm or accessory (i.e., load) away from the user's neck and toward the user's shoulder. Due to the configuration of the sleeve portion, which wraps around the arm of the user, the support sleeve can also shift a portion of the force applied by the weight of the slinged arm or accessory (i.e., load) onto the user's shoulder.

In various embodiments, the load of the arm sling or accessory may be shifted laterally, away from the user's neck and toward a shoulder of the user, by adjusting the slide attachment(s) to capture and hold the shoulder strap away from the user's neck. In other words, one or more of the slide attachments may be moved upwards or downwards on the shoulder strap to reposition or adjust the shoulder strap on the user, shifting the load of the slinged arm or accessory, and reducing a force applied to the user's neck from the shoulder strap.

In an implementation, the slide attachment includes one or more adjustment components. For example a length of the slide attachment may be adjusted via the one or more adjustment components. The user may desire to adjust the length of the slide attachment for better fit, or for positioning and maintaining the shoulder strap in the desired location and position away from the user's neck. For example, the length of the slide attachment may be adjusted (e.g., shortened) to shift the force applied by the weight of the slinged arm or accessory away from the user's neck and toward the user's shoulder.

In various embodiments, the load of the arm sling or accessory may be shifted laterally, away from the user's neck and toward a shoulder of the user, by adjusting a length of the slide attachment. In other words, one or more of the adjustment components may be adjusted to lengthen or shorten a length of the slide attachment, to reposition or adjust the shoulder strap on the user, shifting the load of the slinged arm or accessory on the user to a desired location on the user, and reducing a force applied to the user's neck from the shoulder strap.

In some implementations, the adjustment components are integral to or associated with the slide attachment. In other implementations, the adjustment components are separate but coupled to the one or more slide attachment(s). In one example, the slide attachment may incorporate the adjustment components. For instance, in an implementation, the slide attachment comprises a strip of material looped around the shoulder strap of the sling and coupled to itself via hook and loop fasteners, snaps, or other closure features. In such a case, the adjustment components may include the hook and loop fasteners, multiple snap locations, or the like.

In another example, the slide attachments may be coupled to the sleeve portion via the adjustment components. For instance, the slide attachments may be attached to the sleeve portion using loops, hooks, buckles, d-rings, etc. that provide length adjustment to the sleeve portion as well as attach the slide attachments to the sleeve portion. In some cases, the slide attachment can comprise a fixed closed loop of material. In such cases, the adjustment components provide an adjustable technique for coupling the slide attachment to the sleeve portion.

In various implementations, the support sleeve is comprised of multiple layers. The layers may be combined to provide various desired characteristics for the support sleeve, such as strength, comfort, resilience, utility, weight, and so forth. For instance, outer layers may provide strength and utility while inner layers may provide comfort.

Advantages of the disclosed devices and techniques are varied, and include: better stabilization of the load (e.g., weight) of the slinged arm or accessory; improved weight distribution of the load over the user's shoulder and off the user's neck; beneficial comfort in wearing a sling or carrying a shoulder bag or accessory, particularly over a longer period of time; and a highly functional but inexpensive solution, making it accessible to greater numbers of users. Other advantages may also be appreciated by one skilled in the art.

Techniques and devices are discussed with reference to example arm slings illustrated in the figures. However, this is not intended to be limiting, and is for ease of discussion and illustrative convenience. The techniques and devices discussed may be applied to various sling devices, and the like, arranged to be worn in any of various arrangements, for supporting, stabilizing, or otherwise restricting a user's limb, and remain within the scope of the disclosure. Further, the techniques and devices discussed may also be applied to various shoulder bags, purses, duffel bags, luggage, tool carriers, and other accessories that are commonly worn over the shoulder and neck, and particularly in a cross-body arrangement. In alternate implementations, the techniques and devices disclosed may be employed in other ways or with other devices, systems, arrangements, and so forth. For the purposes of this disclosure, the discussion of a support sleeve with a sling device is also intended to represent the use of a support sleeve with the above and all other similarly worn accessories as well.

Implementations are explained in more detail below using a plurality of examples. Although various implementations and examples are discussed here and below, further implementations and examples may be possible by combining the features and elements of individual implementations and examples.

### Example Sling Device

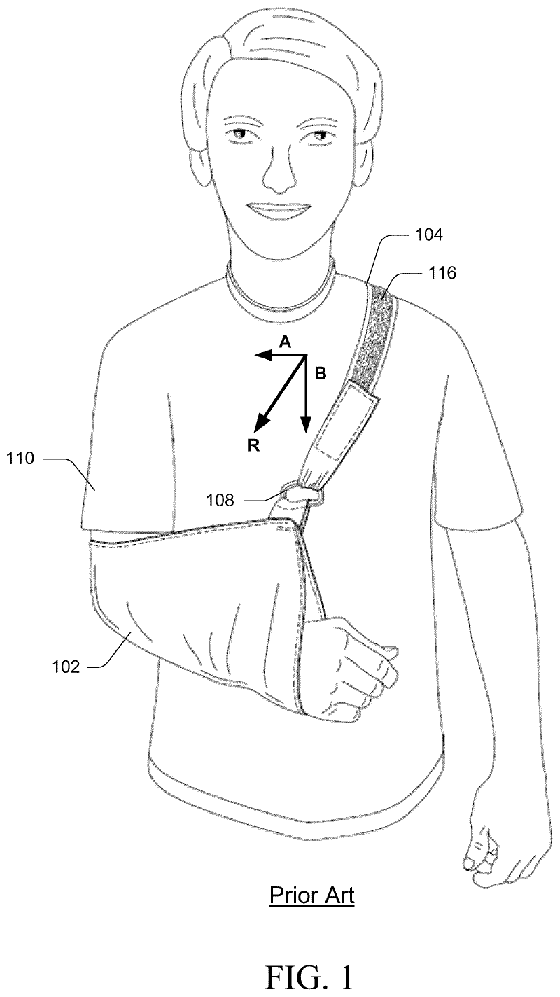

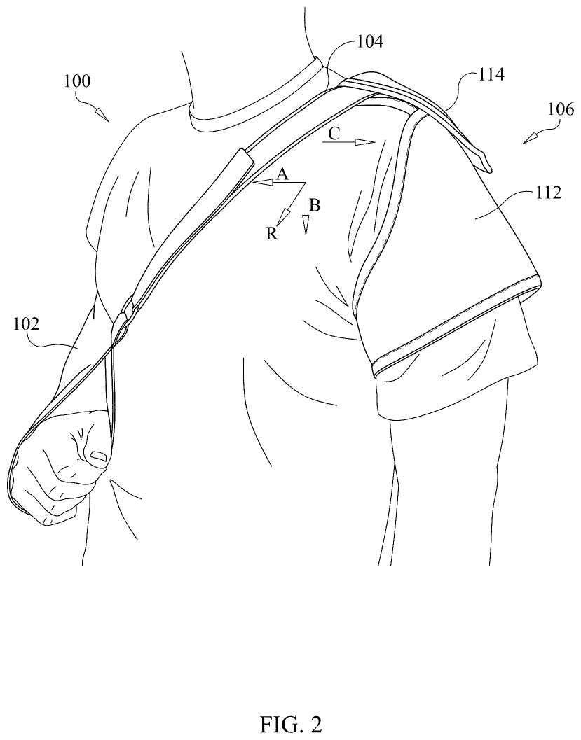

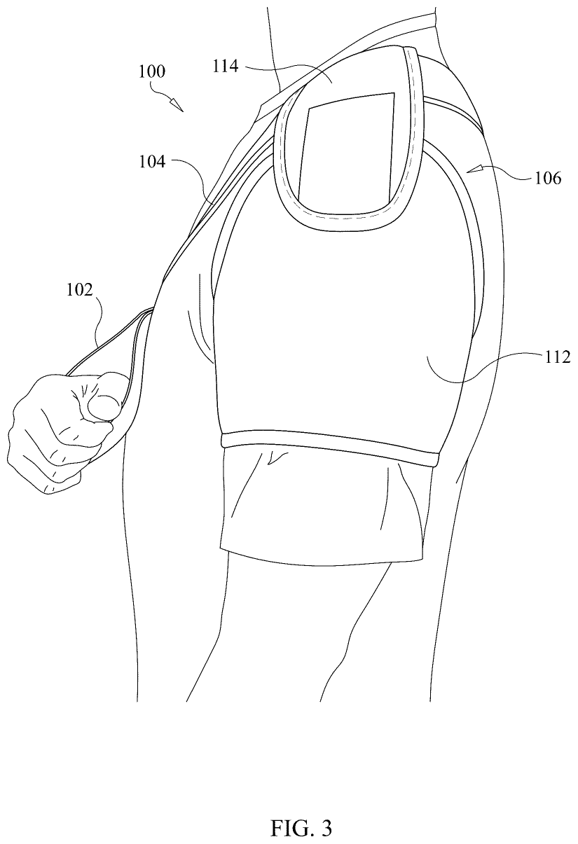

FIG. 1 illustrates a prior art arm sling, which can provide a background and environment for discussion herein. Referring to FIGS. 2-3, an example sling device 100 is shown in non-limiting example configurations. In various embodiments, as shown in FIGS. 2-3, a sling device 100 includes a pocket portion 102, a shoulder strap 104, and a support sleeve 106. In alternate embodiments, the sling device 100 may also include one or more connectors 108 for coupling the pocket portion 102 to the shoulder strap 104 (as shown in FIG. 1, for example). In some examples, the one or more connectors 108 may provide or facilitate adjustment of the shoulder strap 104.

In various implementations, the pocket portion 102 is arranged to cradle a user's arm 110. For example, at least a portion of the user's arm 110 is placed within or is supported by the pocket portion 102. In the implementations, the pocket portion 102 may comprise a pouch, one or more straps, a tube, a sheet of material, or the like. In other implementations, the pocket portion 102 is analogous to a purse, duffel bag, messenger bag, and so forth, with the arm 110 being analogous to the load or weight of the items within the purse, bag, etc.

In an implementation, the shoulder strap 104 is coupled to the pocket portion 102 at one or more locations. In various examples, each end of the shoulder strap 104 is coupled to the pocket portion 102. In one example, a first end of the shoulder strap 104 is coupled to the pocket portion 102 near the wrist/hand opening of the pocket portion 102 and a second end of the shoulder strap 104 is coupled to the pocket portion 104 near the elbow area of the pocket portion 104. In alternate examples, the shoulder strap 104 may be coupled to the pocket portion 102 in various other locations. In each of the examples, the shoulder strap 104 is positioned over the user's shoulder to support a weight of the user's arm 110 (or similar load).

In such an arrangement, as shown in FIG. 1, the force (R) applied by the weight of the slinged arm 110 (or similar load) on the shoulder strap 104 has a downward component (direction B) and an inward component (direction A) toward the user's neck. Accordingly, the inward pulling force (A) applied by the weight of the slinged arm 110 can tend to move the shoulder strap 104 closer to the user's neck as the sling 100 is worn over a short period of time. This shoulder strap 104 movement shifts increasingly more of the weight of the slinged arm 110 to the user's neck, and can cause the user neck pain, neck injury, and related discomfort.

Referring to FIGS. 2 and 3, in various embodiments, a support sleeve 106 is arranged to be coupled to the shoulder strap 104 and also wrapped around a portion of the user's arm. The support sleeve 106 is moveably coupled at one or more points to the shoulder strap 104. As shown at FIGS. 2 and 3, by capturing the shoulder strap 104, the support sleeve 106 applies an outward lateral force (C) that reduces or eliminates the inward component (A) of the force (R) applied by the weight of the slinged arm 110 on the shoulder strap 104. Thus, the support sleeve 106 positions and maintains the shoulder strap 104, and thus the downward component (B) of the weight of the slinged arm 110, at a preferred or desired location (on the user's shoulder, for example). Positioning and maintaining the shoulder strap 104 in the desired location and position can shift the load (i.e., the force (B) or (R)) of the slinged arm 110 off of and away from the user's neck.

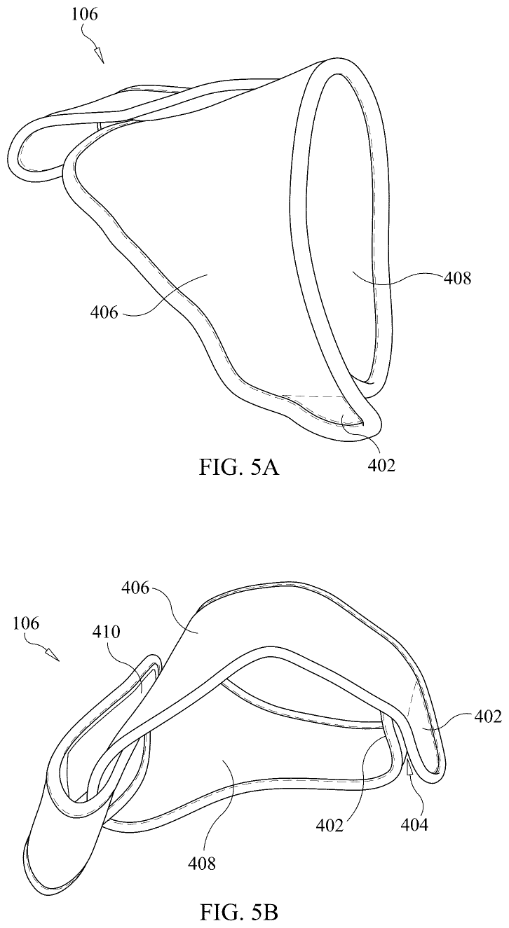

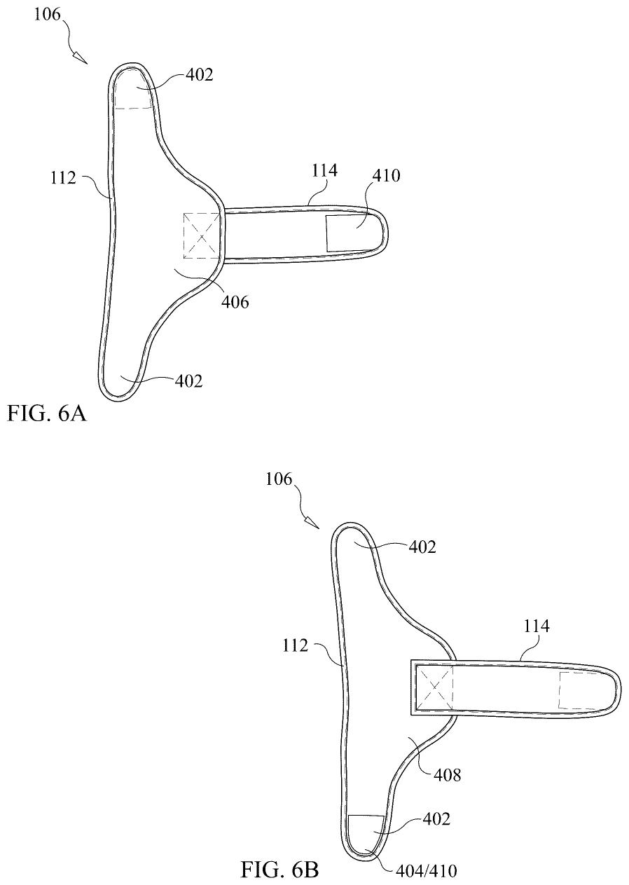

Referring to FIGS. 2-11, in various implementations, the support sleeve 106 includes a sleeve portion 112 and one or more slide attachments 114. In various embodiments, the sleeve portion 112 is arranged to be wrapped around a portion of a user's arm opposite the slinged arm or load.

In some examples, the sleeve portion 112 can comprise a closed or fixed loop or tube (cylinder) of material that can be slid onto a user's arm. In other examples, the sleeve portion 112 may be comprised of a substantially flat strip of textile material that can be wrapped around the arm of the user and temporarily coupled together (using re-closable fasteners, for instance) to form a tube-like sleeve.

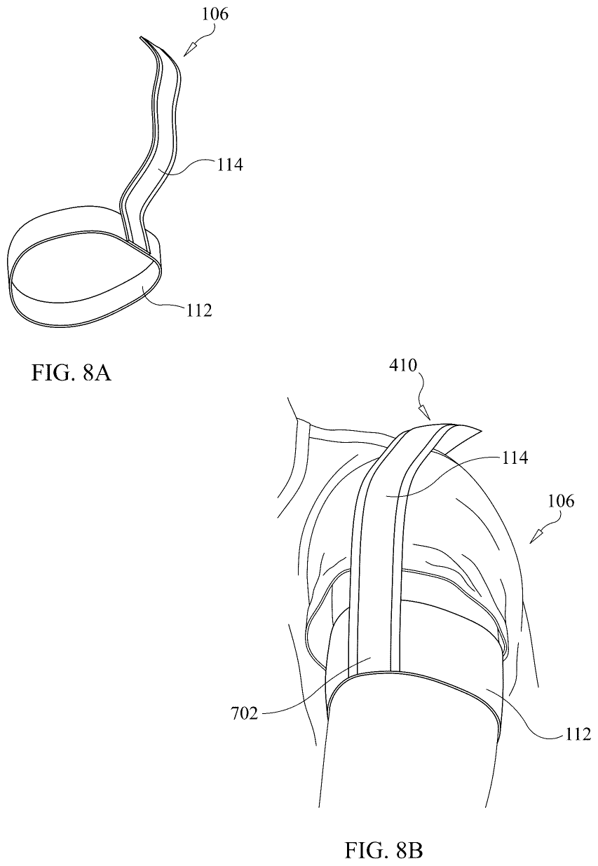

In either case, the sleeve portion 112 can be substantially narrow (such as a one or two inch strip of material, for example) (see FIGS. 8A and 8B, for example) or wider (a couple of inches wide to 5 to 6 inches wide, for example) (see FIGS. 2-7C and 9A-11, for example). Of course, alternate examples can be narrower or wider. The tube-like sleeve portion 112 can be figured or shaped to conform to the user's body (e.g., the upper and/or lower edges of the tube can be configured in a curved shape to fit the upper arm of a user) or the sleeve portion 112 can be a basic cylinder in some cases.

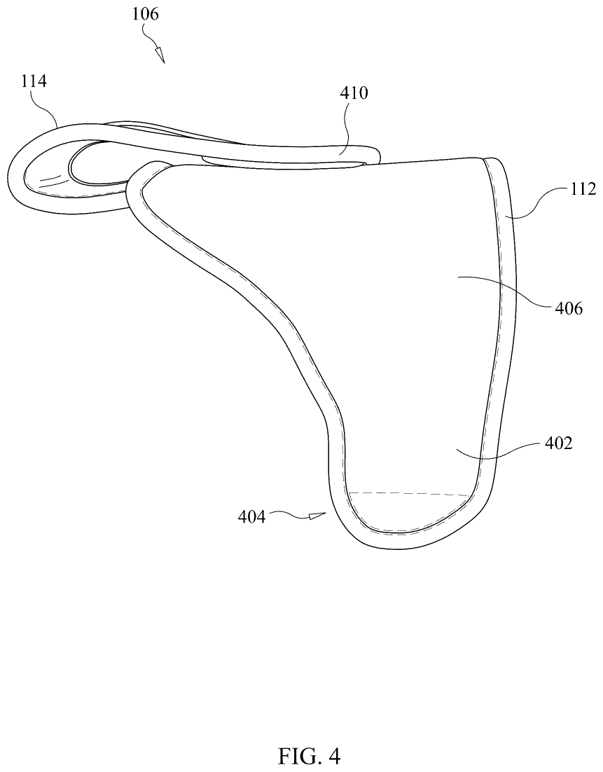

In another example, as shown at FIGS. 4-6B, the sleeve portion 112 may be comprised of a substantially flat section of textile material having “wings” 402 that can be wrapped around the arm of the user and coupled together to form a tube-like sleeve. The flat section or strip of textile material forming the sleeve portion 112 can be sized to fit a variety of persons. For instance, an adult sized sleeve 112 may be approximately 15 to 18 inches in length in some examples when laid flat. In other examples, the sleeve 112 can comprise a flat section or strip of textile material that is longer or shorter, to accommodate the diameter of the user's arm, or a range of users' arms. For instance, the sleeve portion 112 or the entire support sleeve 106 can be formed in various sizes to fit users of different sizes (e.g., multiple children sizes, youth sizes, adult sizes, etc.). In various embodiments, the overall shape of the sleeve portion 112 or the support sleeve 106 may be similar for the different sizes, but sized according to the fit desired.

The wings 402 of the sleeve portion can have couplers 404 (such as snaps, clips, hooks, hook and loop, etc.) disposed thereon for coupling the wings 402 together. In an embodiment, as shown at FIGS. 4-6B, the couplers 404 at the wings 402 can comprise hook and loop fastener material at corresponding parts of the wings 402 for coupling the wings 402 together.

Also referring to FIGS. 4-6B, in some embodiments, the support sleeve 106 may be comprised of multiple layers. The layers may be combined to provide various desired characteristics for the support sleeve 106, such as strength, comfort, resilience, utility, weight, and so forth. For instance, an outer layer 406 may provide strength and utility while an inner layer 408 may provide comfort. Additional layers may also be present between the outer layer 406 and the inner layer 408. Alternately or additionally, one or both of the outer layer 406 and the inner layer 408 may be comprised of multiple layers that are stacked, laminated, glued, stitched, or otherwise coupled together.

In one embodiment, as shown at FIGS. 4-6B, the outer layer 406 of the support sleeve 106 can comprise one of the components of hook and loop fastener (e.g., the “loop” material, or a like material). In the embodiment, the other component (e.g., the “hook” material, or a like material) can be integral to or attached to the end of a wing 402 and the end of a slide attachment 114 (see 410). In such an embodiment, the wing 402 with the hook material can be looped onto the top side of the other wing 402 and the end of the slide attachment 114 can be looped onto the top surface of the slide attachment 114 or the sleeve portion 112.

In various embodiments, the sleeve portion 112 can comprise one or more or a combination of resilient elastic or semi-elastic materials (e.g., spandex, rubber, synthetic rubber, polymers, or the like), one or more non-elastic materials (e.g., a natural fiber material, a nylon material, a para-aramid or aramid material, a fiberglass material, a metallic material, an animal hide, or the like), or combinations of the above. For example, the sleeve portion 112 may be semi-elastic, for comfort and to apply the outward lateral force (C) on the shoulder strap 104. In other examples, the sleeve portion 112 may be generally non-elastic, and may apply the outward lateral force (C) by pushing and/or pulling on the shoulder strap 104.

In some alternate embodiments, the sleeve portion 112 comprises a semi-flexible or non-flexible material (e.g., plastic, composite, fiberglass, metal, etc.), forming a semi-rigid or rigid band or cuff. For example, in the embodiments, the sleeve portion 112 may be molded, stamped, or otherwise formed. In such examples, the sleeve portion 112 may also apply the outward lateral force (C) by pushing and/or pulling on the shoulder strap 104.

In various implementations, as shown in the figures, the support sleeve 106 includes a slide attachment 114 coupled to or integral with a first part of the sleeve portion 112. The slide attachment 114 may be stitched, glued, or otherwise permanently fastened to the sleeve portion 112. In the implementations, the slide attachment 114 is slideably coupled to the shoulder strap 104 of the sling 100 (or accessory), such that the slide attachment 114 has freedom to slide with respect to the shoulder strap 104. In other words, the slide attachment 114 can be looped around the shoulder strap 104 and (removably) fastened to itself (e.g., with hook and loop, snap(s), button(s), hook(s), and so forth).

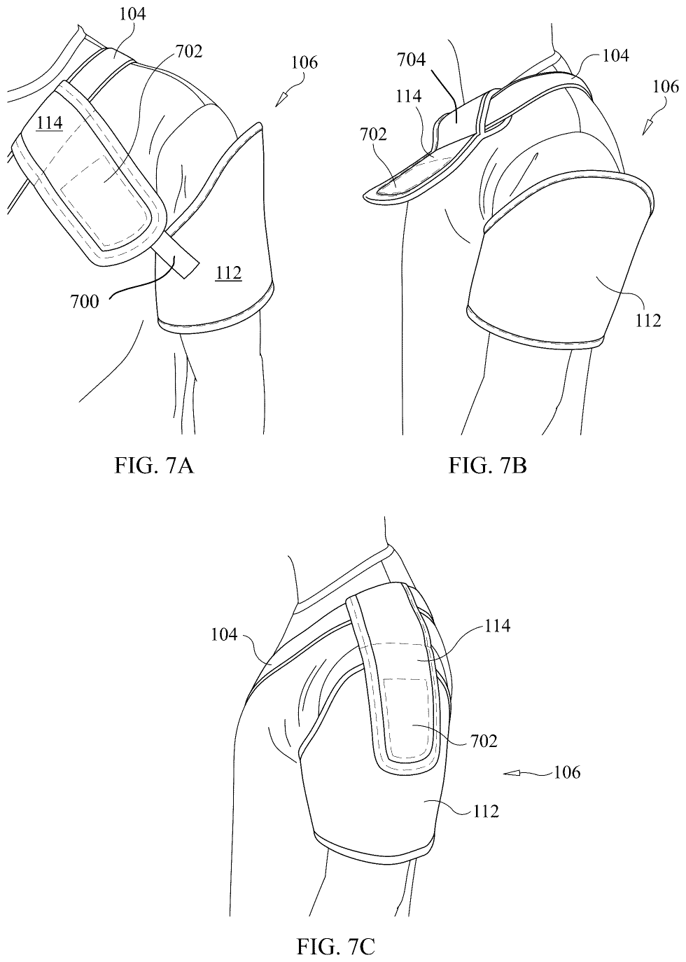

Referring to FIGS. 7A-7C, in some embodiments, the slide attachment 114 is a separate component from the sleeve portion 112, and can be removably coupled to the sleeve portion 112 using fasteners such as hook and loop, snap(s), button(s), hook(s), and so forth. As shown at FIG. 7A, the slide attachment 114 can include a tab 700 configured to attach the slide attachment 114 to the sleeve portion 112. The tab 700 can include the fasteners (or one side of the fasteners—with the other side being on the sleeve portion 112 or the slide attachment 114). The tab 700 can also be adjustable in length, based on a technique used to couple the tab 700 to the slide attachment 114 (e.g., with a clasp, D-ring, hook and loop, etc.). For instance, the tab 700 can comprise a length of textile, polymer, metal, composite, or the like.

In various implementations, the support sleeve 106 includes a first adjustment component 702 configured to couple the slide attachment 114 to the sleeve portion 112. In the implementations, the first adjustment component 702 is arranged to change a length of the slide attachment 114 (from the sleeve portion 112 to the end of the slide attachment 114 that is wrapped around the strap 104). For example, the slide attachment 114 or the sleeve portion 112 may be fed through a portion of the adjustment component 702, and may be adjusted in length by moving the material of the slide attachment 114 or the sleeve portion 112 through or with respect to the adjustment component 702. In other implementations, the first adjustment component 702 is arranged to removably attach the slide attachment 114 to the sleeve portion 112 in an adjustable manner. For example, the first adjustment component 702 may be arranged to attach the slide attachment 114 to the sleeve portion 112 for facilitating the user in putting on the support sleeve 106 or the sling device 100 and may be arranged to detach the slide attachment 114 from the sleeve portion 112 for facilitating the user in taking off the support sleeve 106 or the sling device 100. Additionally, the adjustment component 702 can allow the user to choose the placement of the slide attachment 114 relative to the sleeve portion 112. This can allow the user to adjust the length of the slide attachment 114 and the location of attachment to the sleeve portion 112, for optimal comfort and effectiveness.

In an embodiment, the first adjustment component 702 comprises a hook and loop fastener (or one side of the hook and loop, with the other side on the sleeve portion 112), a buckle, a d-ring, a pair of cinch rings, a snap device, a clip device, a hook device, a clasp device, a combination of the above, or various other components, allowing the user to shorten or lengthen a length of the slide attachment 114, and/or to allow the user to attach and detach the slide attachment 114 to and from the sleeve portion 112.

As also shown at FIGS. 7A-7C, the slide attachment 114 can include a loop 704 at a proximal end of the slide attachment, where the loop 704 may be permanently closed or temporarily (e.g., user-openable) closed. The loop 704 can be slid over the strap 104 to capture the strap 104. The distal end of the slide attachment 114 can be removably attached to the sleeve portion 112.

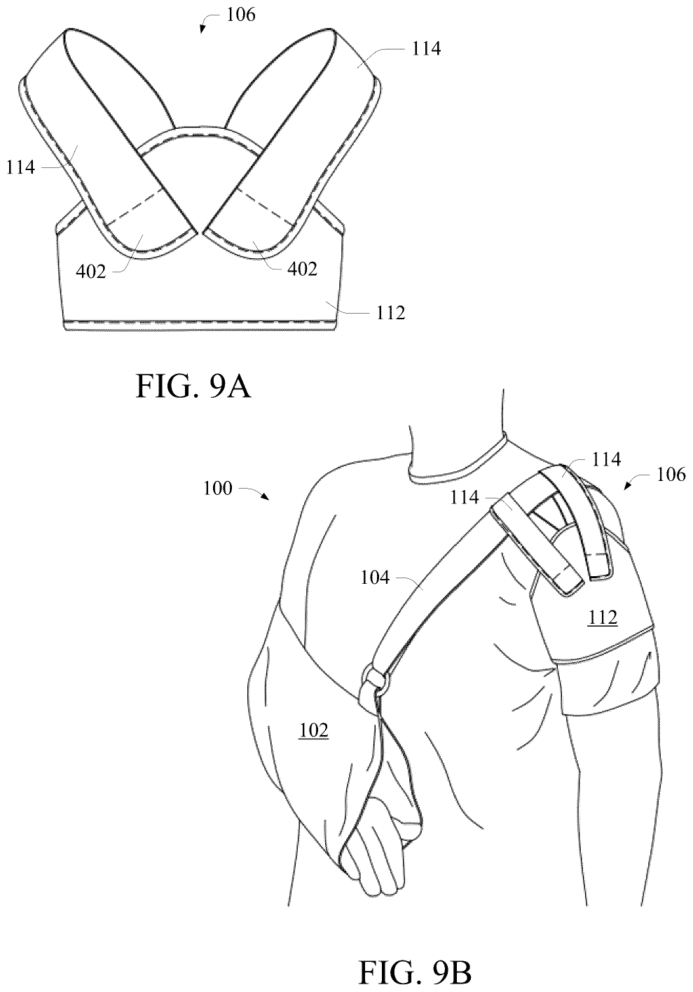

In alternate implementations, as shown at FIGS. 9A and 9B, the support sleeve 106 can include multiple or additional slide attachments 114 coupled to parts of the sleeve portion 112. In the implementations, the multiple or additional slide attachments 114 can also be slideably coupled to the shoulder strap 104 of the sling 100 (or accessory), such that the multiple or additional slide attachment(s) 114 have freedom to slide with respect to the shoulder strap 104. The multiple or additional slide attachment(s) 114 can provide multiple locations to pull on the strap 104 and provide additional control regarding the outward forces on the strap 104. For instance, the multiple slide attachments 114 can be disposed at locations along the strap 104 for optimizing comfort to the wearer.

In an implementation, the slide attachment 114 may be held in the desired location on the shoulder strap 104 via a temporary fastener 444. For example, once the slide attachment 114 is positioned, re-positioned, or adjusted, the slide attachment 114 may be temporarily fixed in place with the temporary fastener 444. In various embodiments, the temporary fastener 444 comprises hook and loop fastener, adhesive, snaps, hooks, buttons, or the like. Accordingly, one or more surfaces (e.g., an inside surface, for example) of the slide attachment 114 may include a portion of the temporary fastener 444 (e.g., the hook or loop material, one half of a snap device, a catch for a hook device, a button hole, etc.).

In any form, the slide attachment 114 can include padding, and particularly padding on the portion of the slide attachment 114 that is configured to be against the body of the user. The addition of padding on the slide attachment 114 can also help to reduce fatigue from carrying the load being worn.

In another embodiment, the slide attachment 114 comprises a loop or tube comprised of a material such as metal, plastic, fabric, animal hide, or the like. In further embodiments, the slide attachment 114 comprises an elliptical ring, a d-ring, a polygonal ring, a strap of material having a hook and loop fastener thereon, or various other components, allowing the slide attachment 114 to slide with respect to the shoulder strap 104.

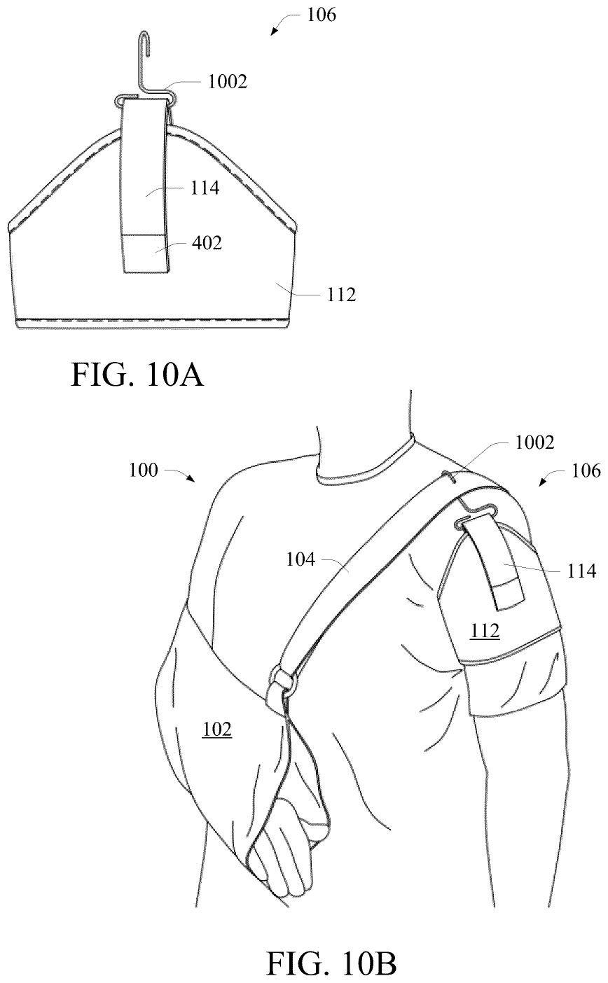

Further, as shown at FIGS. 10A and 10B, the slide attachment 114 can comprise or include a hook device 1002 in addition to one or more slide attachments 114 or in place of other slide attachments 114. The hook device 1002 can be comprised of metal, plastic, composite, or other materials. The hook device 1002 can be permanently or removably coupled to the sleeve portion 112 and arranged to hook and capture the strap 104. In some cases, the hook device 1002 comprises a wide strip of rigid material so as to lay flat on the strap 104 and on the user's shoulder. The wide strip of rigid material may be between 1/2 inch and 1 inch wide in some cases. In alternate examples, the wide strip of rigid material of the hook device 1002 may be narrower or wider.

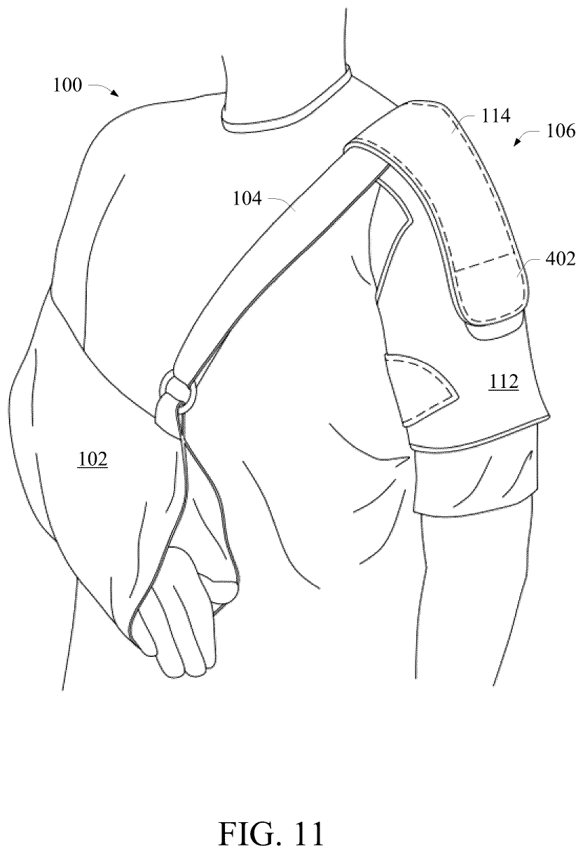

Referring to FIG. 11, in some examples, the slide attachment 114 is integral to the sleeve portion 112. For instance, in one case, the support sleeve 106 comprises a single component (that may be comprised of multiple layers), where one portion of the component is wrapped around the user's arm and another portion of the component is wrapped around the strap 104. Each portion of the support sleeve 106 can be attached back on itself or on another portion of the support sleeve 106 as shown. In some cases, the single component support sleeve 106 is convenient, as there are no separate components that can get separated from other components of the support sleeve 106.

In the various embodiments discussed, as well as other embodiments also contemplated, the slide attachment 114 of the support sleeve 106 constrains or puts tension on the shoulder strap 104 and thereby shifts the load ((R) or (B)) of the arm sling or accessory laterally, away from the user's neck and toward a shoulder of the user (in the direction (C)—see FIG. 2). In the embodiments, the slide attachment 114 of the support sleeve 106 reduces or eliminates a force (A) applied to the user's neck from the shoulder strap 104 by introducing an opposite force in the direction (C).

The examples shown in FIGS. 1-11 and described herein are not intended to be limiting. In alternate implementations, a support sleeve 106 or the sling device 100 may have other arrangements or configurations, and may be used with additional components to reduce or eliminate forces on the neck of the user when the support sleeve 106 is wrapped around the user's arm.

The components and techniques discussed herein with respect to the support sleeve 106 are intended to be used in the production of new support sleeves 106 or sling devices 100 or in the retro-fitting of existing sling devices. In various embodiments, existing sling devices may be upgraded or re-fitted with one or more of the components (e.g., a support sleeve 106), and/or using one or more of the described techniques either individually or in various combinations. In an embodiment, the described components and techniques result in a support sleeve 106 capable of interchangeably coupling to various types of sling devices, or the like. Further, such modified sling devices may be configured and/or adjusted for various user wearing options and locations.

In various implementations, additional or alternative components, techniques, sequences, or processes may be used to implement the techniques described herein. Further, the components and/or techniques may be arranged and/or combined in various combinations, while resulting in similar or approximately identical results. It is to be understood that a support sleeve 106 may be implemented as a stand-alone device or as part of a system (e.g., integrated with other components to form a sling device 100, as described above).

### CONCLUSION

While various discreet embodiments have been described throughout, the individual features of the various embodiments may be combined to form other embodiments not specifically described. The embodiments formed by combining the features of described embodiments are also within the scope of the disclosure.

What is claimed is:

1. An apparatus, comprising:
a sleeve portion arranged as a tube, the sleeve portion configured to be wrapped around a portion of an upper arm of a user; and
a slide attachment integral to or coupled to an upper part of the sleeve portion, and disposed at a shoulder of the user, the slide attachment arranged to be slideably coupled to a shoulder strap of an arm sling or an accessory bag such that the slide attachment applies tension to the shoulder strap in a predetermined direction, which pulls the shoulder strap away from a neck of the user.

2. The apparatus of claim 1, further comprising one or more adjustment components configured to adjust a length of the slide attachment.

3. The apparatus of claim 1, wherein the sleeve portion includes one or more wings configured to be wrapped around the upper arm of the user.

4. The apparatus of claim 3, wherein the wings include one or more fasteners or couplers arranged to couple the wings to each other or to a surface of the sleeve portion.

5. An apparatus, comprising:
a sleeve portion arranged to be wrapped around a portion of a user's arm; and
a slide attachment integral to or coupled to the sleeve portion, the slide attachment arranged to be slideably coupled to a shoulder strap of an arm sling or an accessory bag such that the slide attachment applies tension to the shoulder strap in a predetermined direction.

6. The apparatus of claim 5, wherein the slide attachment comprises a first slide attachment, and further comprising a second slide attachment integral to or coupled to the sleeve portion, the second slide attachment arranged to be slideably coupled to the shoulder strap of the arm sling or the accessory bag such that the second slide attachment applies tension to the shoulder strap in a second predetermined direction . . .

7. The apparatus of claim 5, wherein the apparatus is comprised of multiple layers.

8. The apparatus of claim 7, wherein an outer layer of the multiple layers comprises one of a hook or a loop fastener material.

9. The apparatus of claim 5, wherein the sleeve portion comprises a tube.

10. The apparatus of claim 5, wherein the slide attachment is integral to or permanently coupled to the sleeve portion.

11. The apparatus of claim 5, wherein the slide attachment comprises a loop arranged to capture the shoulder strap.

12. The apparatus of claim 5, wherein the slide attachment comprises a hooked component.

13. The apparatus of claim 5, wherein the slide attachment is removably coupled to the sleeve portion.

14. The apparatus of claim 5, wherein the slide attachment is arranged to pull the shoulder strap away from the user's neck.

15. The apparatus of claim 5, wherein the slide attachment has freedom to slide with respect to the shoulder strap.

16. The apparatus of claim 5, wherein the slide attachment includes at least one fastener component arranged to fix the slide attachment to a portion of the shoulder strap.

17. An arm sling assembly, comprising:
a pocket portion to cradle a first arm of a user;
a shoulder strap coupled at first and second ends of the shoulder strap to the pocket portion, and positioned over a shoulder of the user to support a weight of the first arm of the user; and
a support sleeve moveably coupled to the shoulder strap and arranged to pull the shoulder strap away from a neck of the user, the support sleeve comprising:
a sleeve portion arranged to be wrapped around a portion of a second arm of the user; and
a slide attachment coupled to the sleeve portion, the slide attachment arranged to be slideably coupled to the shoulder strap such that the slide attachment has freedom to slide with respect to the shoulder strap and applies tension to the shoulder strap in a predetermined direction.

18. The arm sling assembly of claim 17, further comprising one or more adjustment components coupled to the slide attachment and arranged to change a length of the slide attachment, the one or more adjustment components selected from the group comprising: a hook and loop fastener, a buckle, a d-ring, a pair of cinch rings, and a snap.

19. The arm sling assembly of claim 17, wherein the slide attachment comprises an extension of the sleeve portion, disposed at the shoulder of the user, and looped over or around the shoulder strap.

20. The arm sling assembly of claim 17, wherein the slide attachment is slideably coupled to the shoulder strap via a hook device, the hook device being permanently or removably coupled to the slide attachment.
